(19) United States
(12) Patent Application Publication
(10) Pub. No.: US 2022/0257401 A1
(43) Pub. Date: Aug. 18, 2022

(54) CLOSURE FOR AN ORTHOSIS

(71) Applicant: THUASNE DEUTSCHLAND GmbH, Großburgwedel (DE)

(72) Inventor: Herbert SCHÖBEL, Burgwedel (DE)

(21) Appl. No.: 17/625,615

(22) PCT Filed: Jun. 15, 2020
(86) PCT No.: PCT/EP2020/066457

§ 371 Date: Jan. 7, 2022

(30) Foreign Application Priority Data
Jul. 8, 2019 (DE) 20 2019 103 735.0

(51) Int. Cl. A61F 5/01

(52) U.S. Cl. CPC A61F 5/0102

(57) ABSTRACT

The invention relates to a closure for an orthosis, in particular a quick closure for an orthosis, having an attachment part (1) and a retaining portion (14), wherein the attachment part (1) is detachably fastened to the retaining portion (14). The attachment part (1) has at least two tuck-in flaps (3, 3), which are connected to each other at one first end on a base (2) of the attachment part (1) and delimit a receptacle region (5) of the attachment part (1) at two opposite sides. The retaining portion (14) is designed such that, when the attachment part (1) is fixed thereto, it is held in the receptacle region (5) between the two tuck-in flaps (3, 3′). The retaining portion (14) is part of a frame or a framework (26) or a rail or another part of an orthosis and has at least one recess (15). One of the tuck-in flaps (3, 3′) has at least one latching element (6) which engages in the recess (15) forming a indentation when the attachment part (1) is slid onto the retaining portion (14).

The invention relates to a closure for an orthosis, in particular a quick-release closure for an orthosis, comprising an attachment part and a retaining portion, wherein the attachment part can be detachably fastened to the retaining portion.

Corresponding closures for orthoses are used primarily for those orthoses that require frequent donning and doffing, for example, when the orthosis is to be worn only when load is applied on a respective body part. The retaining portion is usually an attachment part arranged on or on a base body of the orthosis, which cooperates with the attachment part in a clip-like manner, wherein the attachment part can be latched in or on the receiving part designed as a retaining portion. The attachment part is furthermore frequently associated with a movable and/or adjustable part of the orthosis with which the orthosis can be fixed to the respective body part. In the case of known orthoses, the attachment part and the receiving part of the closure are each fitted parts which are fastened to or on the actual orthosis or the base body of the orthosis. Fastening the closure can then be carried out by rivets, textile welding or seam connections, among other things. However, since the known closures are mostly arranged superficially on the base body of the orthosis, they can more easily tear out or otherwise become loose.

It is therefore an object of the invention to provide a closure for an orthosis which is better integrated into the rest of the orthosis and thus ensures a particularly secure connection.

This object is achieved with a closure with the features of claim 1 and an orthosis with the features of claim 13. The closure for an orthosis, in particular the quick-release closure for an orthosis, comprising an attachment part and a retaining portion, wherein the attachment part can be detachably fastened to the retaining portion, is characterized according to the invention in that the attachment part has at least two tuck-in flaps which are connected to one another at a first end on a base of the attachment part and delimit a receptacle region of the attachment part with respect to two opposite sides, that the retaining portion is designed such that, when the attachment part is fastened thereto, it is held in the receptacle region between the two tuck-in flaps, in particular is clamped in place in the receptacle region between the two tuck-in flaps, that the retaining portion is part of a frame or a framework or a rail or another part of an orthosis, that the retaining portion has at least one recess, that at least one of the tuck-in flaps has at least one latching element, and that, when the attachment part is slid onto the retaining portion, the latching element engages in the recess forming an indentation.

Due to the fact that the attachment part can be directly connected to the frame, the framework or the rail or another part of the orthosis via the tuck-in flaps and the retaining portion, it is achieved in a simple manner that the closure is integrated into the orthosis in the best possible way. The attachment part engages directly with “load-bearing parts” of the orthosis, such as the frame, the framework, or the rail or another part, in particular a dimensionally stable part. As a result, a particularly secure hold of the closure on the orthosis and of the orthosis on a respective body part is achieved. By using the frame, the framework or the rail of the orthosis as the retaining portion, a reduction in the number of parts required for the closure is also achieved, since a separate receiving part of the closure for the attachment portion is eliminated. The two tuck-in flaps further enable the attachment part to be easily guided, so that the closure can be handled even by untrained persons.

The latching element can be a latching lug projecting in a simple manner into the receptacle region. In a further configuration, the latching element, in particular the latching lug, has at least one projection pointing in the direction of the base of the attachment part, wherein an angle of less than 90° is formed between mutually facing surfaces of the tuck-in flap on which the latching element is arranged and of the latching element itself. By means of the projection pointing in the direction of the base of the attachment part, greater wear and stress on the latching element is counteracted in a simple manner. Even if the projection wears increasingly by opening and closing the closure and therefore the angle between the facing surfaces of the tuck-in flap and the latching element increases, it is still possible to ensure secure latching of the closure over a normal period of use.

In order to achieve improved guidance of the attachment part when closing the closure, it can further be provided that the latching element with its rear side facing the oppositely arranged tuck-in flap becomes increasingly wider in the direction of the base and forms a wedge. The rear side of the latching element has a flatter angle to the tuck-in flap on which the latching element is arranged than the aforementioned surface facing the tuck-in flap to the projection in order to simplify sliding the attachment part with the latching element onto the retaining portion.

To ensure easy opening of the closure and thus improve manageability, it is further provided that at least one of the tuck-in flaps is designed as a grip tab at its free end opposite the first end. In a further configuration, the grip tab is a portion of the attachment part that is bent outwards, i.e. away from the receptacle region of the tuck-in flaps. This bent portion facilitates gripping of the tuck-in flap when the tuck-in flap is bent outwards. When the tuck-in flap is bent open, an increase in the distance between the tuck-in flaps to each other and thus an enlargement of the receptacle region between the tuck-in flaps is achieved, so that the latching element can slide out of the recess. According to an advantageous configuration, the grip tab is therefore arranged on the tuck-in flap that also has the latching element.

Furthermore, according to a refinement, at least one of the tuck-in flaps is longer than the other tuck-in flap. Such a longer, protruding tuck-in flap portion can serve as an orientation aid or insertion aid when fastening the attachment part. A user places this extended tuck-in flap section on the retaining portion and has the attachment part directly positioned in terms of the height.

In particular, the tuck-in flap with the grip tab can also be designed to be longer than the tuck-in flap without the grip tab in order to provide a person using the closure or the orthosis with a haptic and visual orientation aid during use.

According to a refinement, the attachment part is associated with the part of an orthosis that serves to fix the orthosis to a body part. Such a part of an orthosis is, for example, a loop, a strap, a Velcro strip or the like, each of which is adjustable and enables precise adjustment of the orthosis to a particular body part of a patient. However, the enumeration is not exhaustive.

Now, in order to connect the attachment part to the aforementioned part or parts for fixing the orthosis, such as a loop, a strap, a Velcro strip or the like, the attachment part can comprise at least one loop receptacle. Such a combination of attachment part and loop, strap, Velcro strip or the like has the advantage that the orthosis can be specially adjusted to a patient by skilled personnel when donning it for the first time, and the patient subsequently only has to release the closure. The settings made once are thus retained even if the closure is opened and closed multiple times.

To ensure that the closure is subjected to as little stress as possible while the orthosis is being worn, it is provided according to a refinement that starting from the base, the loop receptacle is arranged opposite the receptacle region for the retaining portion, in particular arranged at an opposite end to the receptacle region for the retaining portion. The indentation formed between the recess of the retaining portion and the latching element of the tuck-in flaps is thus as far away as possible from the loop receptacle and from forces acting on the loop receptacle. Unwanted forcing the tuck-in flap open with the latching element is thus counteracted.

A further reduction of the forces acting on the closure, in particular forces acting on the connection between the retaining portion and the tuck-in flaps, can be ensured according to a refinement in that the loop receptacle is connected to the base of the attachment part via a movable connection. Depending on the design of the closure, the movable connection can be made in different ways. In the case of a one-piece attachment part, the movable connection according to a refinement can be, for example, a flexible web, and in the case of a two-piece or multi-piece attachment part, the movable connection can be, for example, a hinged connection.

In this case, for example, the flexible web can be characterized by the fact that it is made of a thinner and thus more flexible material than the rest of the attachment part.

The hinged connection can be formed in a simple manner by a pivot axle associated with the base, at which axle the parts of the attachment part are brought together, in particular are pivotably connected to one another. According to advantageous configurations, the hinged connection can either be formed by a quick-release axle fed through the loop receptacle and the base, or the loop receptacle has a portion forming an axle which is pivotably clamped in the base, in particular is clamped in an axle guide of the base.

In order to be able to fix the attachment part to the retaining portion in a simple manner, it is provided according to a refinement that the recess is associated with an edge of the frame, or the framework or the rail or another part, in particular a dimensionally stable part, of the orthosis and an attachment part guide is formed on the edge in the region of the recess. In addition to a correspondingly shaped latching element, in particular one that widens in the direction of the base, the attachment part guide serves to simplify fitting together the attachment part and the retaining portion of the closure. The latching element and the attachment part guide advantageously cooperate in such a manner that the attachment part is centered relative to the recess in the retaining portion.

According to a refinement, the attachment part guide can be formed by a receiving funnel narrowing in the direction of the recess, in particular by a receiving funnel narrowing to the width of the recess. When closing the closure, the latching element is then guided by the receiving funnel and slides in the direction of the recess of the retaining portion. The recess and the receiving funnel are advantageously separated from each other by a retaining web of the retaining portion. In this case, the retaining web also forms the indentation to which the latching element is fixed, in particular latched. To ensure optimum hold of the latching element in the recess, the latching element can have a maximum width that is slightly smaller than the width of the recess, in particular slightly smaller than a maximum width of the recess.

The receiving funnel of the attachment part guide can be formed in a simple manner by bead-like side regions of the retaining portion which, on the sides of the edge facing away from the receiving funnel, are adjoined in each case by respective concave rim portions of the retaining portion. The bead-like side regions extend in the direction of an attachment part to be slid onto the retaining portion. The concavely extending edge of the retaining portion allows the attachment part to be pivoted, wherein the attachment part is delimited in its pivoting range by its width at the base and the radius of the concave edge.

In addition to the latching element, a guide web can be arranged on the tuck-in flap opposite the latching element, which guide web extends from the base in the direction of the latching element. This guide web is adapted to the attachment part guide, in particular the receiving funnel of the attachment part guide, so that the attachment part, which is slid onto the retaining portion and latched, is arranged with the guide web in the receiving funnel between the bead-like side regions of the edge of the retaining portion.

The invention further relates to an orthosis comprising a frame, a framework or a rail, characterized in that it comprises at least one closure described above.

An exemplary embodiment of the invention, from which further features essential to the invention may result, is illustrated in the drawing. In the figures:

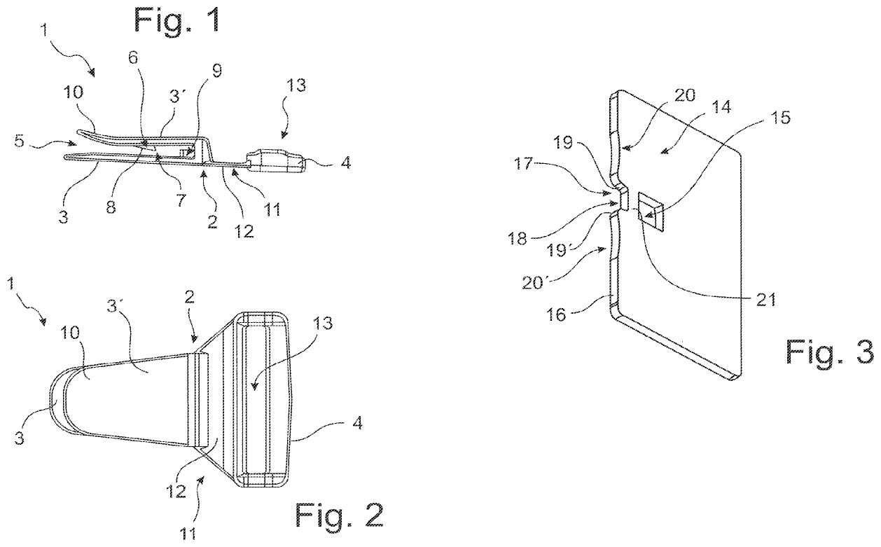

FIG. 1 shows a side view of a first embodiment of an attachment part of the closure according to the invention;

FIG. 2 shows a top view of the first embodiment of the attachment part of the closure according to the invention;

FIG. 3 shows a perspective illustration of a retaining portion of the closure according to the invention;

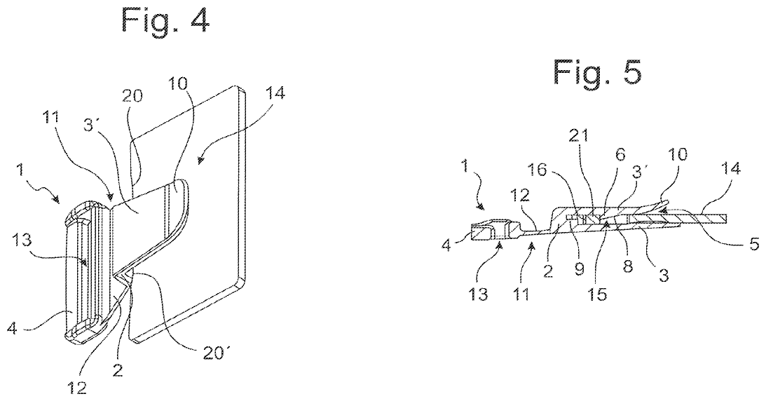

FIG. 4 shows a perspective illustration of the attachment part according to FIG. 1 and FIG. 2 attached to the retaining portion according to FIG. 3;

FIG. 5 shows a cross section through the retaining portion and the attachment part according to FIG. 4;

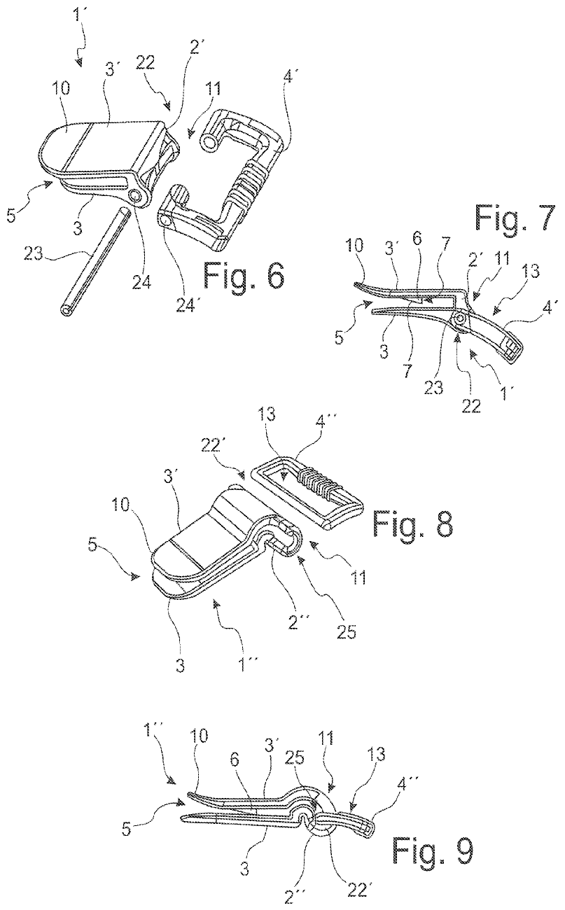

FIG. 6 shows an exploded view of a second embodiment of an attachment part of the closure according to the invention;

FIG. 7 shows a side view of the assembled attachment part according to FIG. 6;

FIG. 8 shows an exploded view of a third embodiment of an attachment part of the closure according to the invention;

FIG. 9 shows a side view of the assembled attachment part according to FIG. 8;

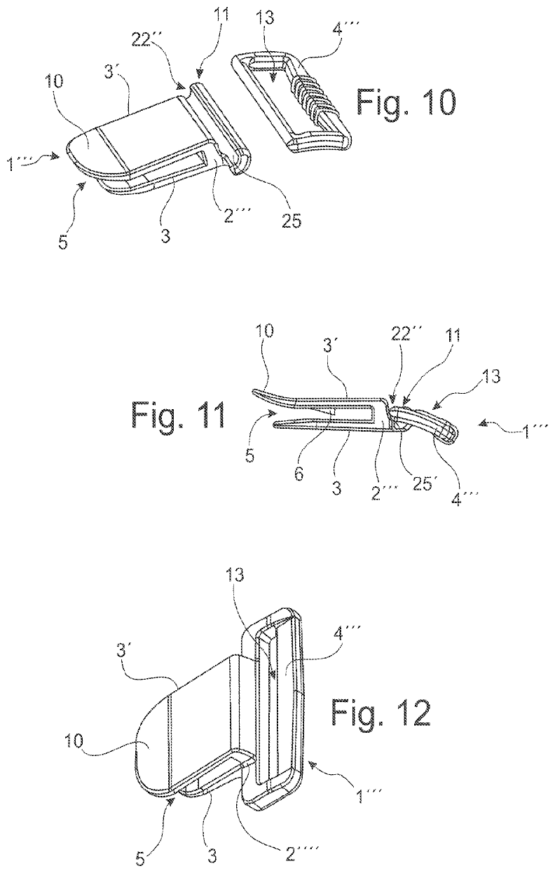

FIG. 10 shows an exploded view of a fourth embodiment of an attachment part of the closure according to the invention;

FIG. 11 shows a side view of the assembled attachment part according to FIG. 10;

FIG. 12 shows a perspective view of a fifth embodiment of an attachment part of the closure according to the invention; and

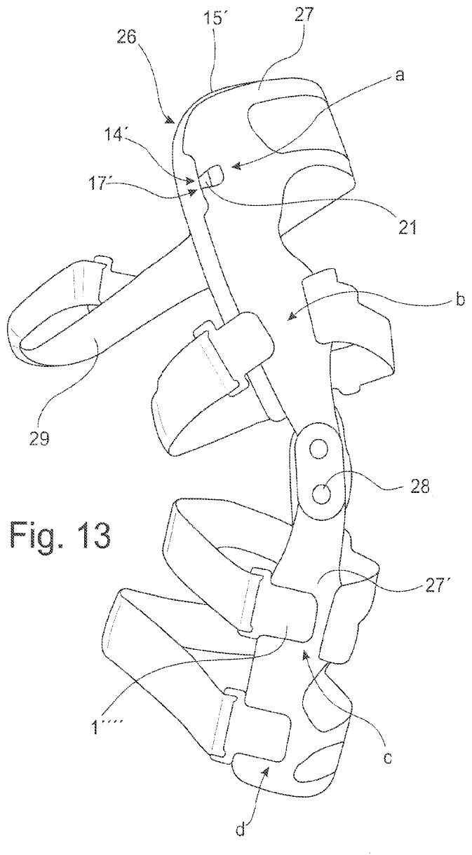

FIG. 13 shows an orthosis according to the invention with a closure according to the invention.

FIG. 1 shows a first embodiment of an attachment part 1. This attachment part 1 has a base 2 which is arranged centrally between two opposite sides of the attachment part 1. Starting from the base 2, two tuck-in flaps 3, 3′ extend to a first side and a loop receptacle 4 extends to a second side. The tuck-in flaps 3, 3′ are aligned parallel to each other and delimit a receptacle region 5 of the attachment part 1. On the sides of the tuck-in flaps 3, 3′ facing each other, the tuck-in flap 3 has a latching element 6. This latching element 6 has a projection 7 pointing in the direction of the base 2, wherein between mutually facing surfaces of the tuck-in flap 3, in particular an inner side of the tuck-in flap 3 on which the latching element 6 is arranged, and of the latching element 6 itself, an angle of less than 90° is formed. With a rear side 8 facing the tuck-in flap 3′, the latching element 6 has an angle with respect to the to the tuck-in flaps 3, 3′ that is substantially flatter than with the surface facing the tuck-in flap 3. Furthermore, a guide web 9 extending from the base 2 in the direction of the latching element 6 is provided on the tuck-in flap 3′. Both the guide web 9 and the latching element 6 with their respective regions protruding furthest with respect to the tuck-in flaps 3, 3′ project into the receptacle region 5 up to a center line formed between the tuck-in flaps 3, 3′.

A grip tab 10 with which the tuck-in flap 3 and thus the receptacle region 5 can be bent open in order to open or release the closure is also provided on the tuck-in flap 3. This grip tab 10 is arranged at a free end of the tuck-in flap 3 opposite the base 2.

The loop receptacle 4 is connected to the base 2 via a movable connection 11 which is designed as a flexible web 12. Furthermore, the loop receptacle 4 has a recess 13 which can be seen in particular from the top view in FIG. 2. This recess 13 serves for the passage of a loop, a strap, a Velcro strip or the like with which an orthosis comprising the closure can then be fixed to a body part.

In FIG. 2, as in the other figures, same parts are designated with the same reference signs as in FIG. 1. Noticeable in FIG. 2 is that the tuck-in flaps 3, 3′ become increasingly narrower towards their free ends and the loop receptacle 4 with the recess 13 has an overall substantially greater width than the tuck-in flaps 3, 3′. In this case, the flexible web 12 has a trapezoidal base surface which, on the one hand, is adapted to the maximum width of the base 2 and, on the other hand, is adapted to the dimension of the loop receptacle 4. In the area of the grip tab 10 at the free end of the tuck-in flap 3, the latter also has a rounded shape which enables ergonomic operation.

FIG. 3 shows a retaining portion 14 formed as a counterpart to the attachment part 1. This retaining portion 14 has a recess 15 which is associated with an edge of the retaining portion 14. The edge 16 of the retaining portion 14 is formed into an attachment part guide 17. The attachment part guide 17 is composed of a receiving funnel 18, bead-like side regions 19, 19′ and concave rim portions 20, 20′ adjoining the side regions 19, 19′. Furthermore, the receiving funnel 18 of the attachment part guide 17 and the recess 15 are arranged relative to one another in such a manner that they are aligned with one another with the receiving funnel 18 narrowing in the direction of the recess 15 to the width of the recess 15. Moreover, the recess 15 and the receiving funnel 18 are separated from one another by a retaining web 21, the retaining web 21 forming an indentation for the latching element 6 when the attachment part 1 is attached.

In the following FIGS. 4 and 5, attachment part 1 and retaining portion 14 are each shown connected to one another, so that FIG. 4 and FIG. 5 show the closed closure. The tuck-in flaps 3, 3′ surround the retaining portion 14 on both sides of the retaining portion 14 which is received in the receptacle region 5.

The sectional view of FIG. 5 further shows in particular how the retaining portion 14 and the attachment part 1 engage in each other. The retaining portion 14 has a thickness that corresponds to the distance between the tuck-in flaps 3, 3′, that is in particular equal to the thickness of the receptacle region 5. The latching element 6 is arranged in the recess 15 and clamped to the retaining web 21. The retaining web 21 is arranged between the latching element 6 and the guide web 9.

A second embodiment of the attachment part 1′ is shown in FIG. 6. This second embodiment differs from the attachment part 1 substantially in that as a movable connection 11, a hinged connection 22 with a quick-release axle 23 is provided, which quick-release axle, in an axle passage 24, 24′, can be fed through the base 2 and at least a part of the loop receptacle 4′ associated with the base 2. The assembled attachment part 1′ is shown in FIG. 7. From this illustration, an ergonomic curvature of the attachment part 1′ becomes clearly visible, wherein both the tuck-in flap 3′ and the loop receptacle 4 are ergonomically curved. In addition, the tuck-in flap 3 is made longer than the tuck-in flap 3′. This longer, protruding tuck-in flap portion serves as an orientation aid or insertion aid when fastening the attachment part 1′. A user places this extended tuck-in flap portion on the retaining portion 14, 14′ and has the attachment part 1′ directly positioned in terms of the height.

FIG. 8 shows a third embodiment of the attachment part 1″. Like the second embodiment of the attachment part 1′, the third embodiment of the attachment part 1″ shows a two-part variant in which the base 2″ with the tuck-in flaps 3, 3′, on the one hand, and the loop receptacle 4″, on the other hand, each form part of the attachment part 1″. Instead of a separate axle or quick-release axle, a portion of the loop receptacle 4″ is designed here as the axle of a hinged connection 22′. This portion of the loop receptacle 4″ is inserted through the receptacle region 5 in an axle guide 25 integrated in the base 2″ as shown in FIG. 9 and is pivotably held in this axle guide 25. For this purpose, a curved recess adjoining the receptacle region 5 is provided as axle guide 25 in the base 2″.

Finally, FIG. 10 and FIG. 11 show a fourth embodiment of the attachment part 1″. This fourth embodiment uses the same loop receptacle 4″ as in FIG. 8 and FIG. 9. In contrast to the previous third embodiment, an axle guide 25′ is provided here as a hinged connection 22″ of the movable connection 11, which is arranged laterally to the base 2″ on the side of the base 2″ facing away from the receptacle region 5. Thus, in the fourth embodiment of the attachment part 1″, the loop receptacle 4″ does not have to be inserted via the receptacle region 5 or through the receptacle region 5.

A fifth embodiment of the attachment part 1″ is shown in FIG. 12. The embodiment according to FIG. 12 is a particularly simple variant without a movable connection between the base 2″ and the loop receptacle 4″. Rather, the loop receptacle 4″ is directly attached to the base 2″.

FIG. 13 shows an orthosis for the treatment of a patient's body part with a total of four closures a, b c, d according to the invention. This orthosis has a framework 26 for stabilizing the body part, which at the same time forms retaining portions 14′ of the closures a, b, c, d. The framework 26 is made up of two parts. The framework 26 is composed of two parts 27, 27′ interconnected by a joint 28, each part 27, 27′ of the framework 26 having two of the closures a, b, c, d and accordingly two retaining portions 14′. An upper part 27 of the framework 26 has the closures a, b and a lower part 27′ of the framework 26 has the closures c, d. Each closure a, b, c, d is associated with a retaining portion 14′ and an attachment part 1″. The attachment parts V″ are each connected via their loop receptacle 4″ to a loop strap 29, the length of which can be adapted to a patient. While the closures c, d are each shown in the closed position, the closures a, b are shown open and partially open, respectively. The structure of the retaining portion 14′ with its attachment part guide 17 and its recess 15′ can be seen particularly well in the case of the closure a shown fully open. This attachment part guide 17′ differs in that it is formed by a bulge along the edge 16 of the framework 26 corresponding to the width of the attachment part 4′. In this case, the recess 15′ is separated from the bulge by a retaining web 21 and arranged centered thereto.

All the features mentioned in the foregoing description and in the claims can be combined in any desired selection with the features of the independent claim. The disclosure of the invention is thus not limited to the combinations of features described or claimed, rather all combinations of features useful within the scope of the invention are to be considered as being disclosed.

1. A closure for an orthosis, in particular a quick-release closure for an orthosis, comprising an attachment part and a retaining portion, wherein the attachment part can be detachably fastened to the retaining portion,
wherein the attachment part has at least two tuck-in flaps which are connected to one another at a first end on a base of the attachment part and delimit a receptacle region of the attachment part with respect to two opposite sides,
wherein the retaining portion is designed such that, when the attachment part is fastened thereto, it is held in the receptacle region between the two tuck-in flaps,
wherein the retaining portion is part of a frame or a framework or a rail or another part of an orthosis,
wherein the retaining portion has at least one recess,
that wherein at least one of the tuck-in flaps has at least one latching element, and
wherein the latching element engages in the recess forming an indentation when the attachment part is slid onto the retaining portion.

2. The closure (a, b, c, d) according to claim 1, wherein the latching element has at least one projection pointing in the direction of the base of the attachment part, wherein an angle of less than 90° is formed between mutually facing surfaces of the tuck-in flap on which the latching element is arranged and of the latching element itself.

3. The closure according to claim 1, wherein the latching element with its rear side facing the oppositely arranged tuck-in flap becomes increasingly wider in the direction of the base and forms a wedge.

4. The closure according to claim 1, wherein at least one of the tuck-in flaps is formed as a grip tab at its free end opposite the first end.

5. The closure according to claim 1, wherein at least one of the tuck-in flaps is longer than the other tuck-in flap.

6. The closure according to claim 1, wherein the attachment part has at least one loop receptacle.

7. The closure according to claim 6, wherein starting from the base, the loop receptacle is arranged opposite the receptacle region for the retaining portion.

8. The closure according to claim 6, wherein the loop receptacle is connected to the base of the attachment part via a movable connection.

9. The closure according to claim 8, wherein in the case of a one-piece or multi-piece attachment part, the movable connection is made via a flexible web and in the case of a two-piece attachment part, is made via a hinged connection.

10. The closure according to claim 1, wherein the recess is associated with an edge of the frame or of the framework or of the rail or of the other part of an orthosis, and an attachment part guide is formed on the edge in the region of the recess.

11. The closure according to claim 10, wherein the attachment part guide is formed by a receiving funnel narrowing in the direction of the recess.

12. The closure according to claim 11, wherein the receiving funnel is formed by bead-like side regions which, on the sides of the edge facing away from the receiving funnel, are adjoined in each case by concave rim portions of the retaining portion.

13. The closure according to claim 1, wherein a guide web is arranged on the tuck-in flap opposite the latching element, which guide web, starting from the base, extends in the direction of the latching element.

14. An orthosis with a frame, a framework or a rail,
wherein the orthosis comprises at least one closure according to claim 1.
